(19) United States
(12) Patent Application Publication
(10) Pub. No.: US 2025/0381245 A1
(43) Pub. Date: Dec. 18, 2025

(54) METHODS AND COMPOSITIONS OF TREATING CANCER BY MODULATING PALMITOYLATION OF FLOTILLIN-1

(71) Applicant: BOARD OF REGENTS, THE UNIVERSITY OF TEXAS SYSTEM, Austin, TX (US)

(72) Inventors: Linda DEGRAFFENRIED, San Antonio, TX (US); Bryan MCCLELLAN, Austin, TX (US)

(21) Appl. No.: 18/878,526

(22) PCT Filed: Jun. 26, 2023
(86) PCT No.: PCT/US2023/069076

§ 371 Date: Dec. 23, 2024

Related U.S. Application Data
(60) Provisional application No. 63/355,246, filed on Jun. 24, 2022.

(51) Int. Cl. A61K 38/17

(52) U.S. Cl. CPC A61K 38/177

(57) ABSTRACT

Cancer metastasis is the leading cause of cancer related death and involves the spreading of the tumor to distant sites throughout the body. However, there are limited therapies which can effectively target this deadly process. One protein which contributes to cancer metastasis is termed flotillin-1. Its expression is associated with metastasis in several solid tumors. It is known that the addition of a fatty acid can alter this protein's biology and stabilize it (“palmitoylation”). It is herein shown that altering this process can lead to a decrease in the stability of the protein. Thus, targeting the addition of this fatty acid can lead to its degradation and be a way to successfully target this metastasis-inducing protein therapeutically.

### REFERENCE TO SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Jun. 26, 2023, is entitled “10046-488WO1_ST26.xml”, and is 21,569 bytes in size.

### CROSS-REFERENCE TO RELATED APPLICATIONS

This application claims benefit of U.S. Provisional Application No. 63/355,246, filed Jun. 24, 2022, which is hereby incorporated herein by reference in its entirety.

### BACKGROUND

Cancer metastasis is the leading cause of cancer related death and involves the primary tumor spreading to distant organ sites (Weigelt, Peterse et al. 2005). Flotillin-1 has multiple published studies demonstrating its role in cancer metastasis (2016, Cao, Cui et al. 2016, Li, Yang et al. 2016, Ou, Liu et al. 2017). However, challenges have arisen in the ability to actually target the protein therapeutically. The addition of the fatty acid palmitate to this protein, termed palmitoylation, has also been implicated in resulting in its stability and expression (Jang, Kwon et al. 2015). There have been studies published previously utilizing a peptide inhibitor to competitively inhibit the palmitoylation process of other proteins involved in cancer immunity, but not Flotillin-1 (Yao, Lan et al. 2019).

What is needed in the art are methods and compositions for targeting flotillin-1 palmitoylation as a therapy for cancer metastasis.

### SUMMARY

The present invention relates to a method of inhibiting flotillin-1 activity, the method comprising decreasing palmitoylation of flotillin-1 in a cell.

The invention also relates to a method of identifying inhibitors of palmitoylation of flotillin-1, the method comprising the steps of: providing flotillin-1; providing one or more enzymes which catalyze palmitoylation of flotillin-1; providing one or more potential inhibitors of flotillin-1 palmitoylation; and detecting inhibition of palmitoylation of flotillin-1 by a potential inhibitor, thereby identifying an inhibitor of palmitoylation of flotillin-1.

The invention further relates to peptides comprising 90% or more identity to any one of SEQ ID NOS: 3-10.

Additional aspects and advantages of the disclosure will be set forth, in part, in the detailed description and any claims which follow, and in part will be derived from the detailed description or can be learned by practice of the various aspects of the disclosure. The advantages described below will be realized and attained by means of the elements and combinations particularly pointed out in the appended claims. It is to be understood that both the foregoing general description and the following detailed description are exemplary and explanatory only and are not restrictive of the disclosure.

### BRIEF DESCRIPTION OF THE FIGURES

The accompanying drawings, which are incorporated in and constitute a part of this specification, illustrate certain examples of the present disclosure and together with the description, serve to explain, without limitation, the principles of the disclosure. Like numbers represent the same elements throughout the figures.

FIG. 1A-B shows (A) membrane binding/localization site, and flotillin gene, along with (B) a specific decoy sequence with a CPPtat leader and a fragment of Flotillin-1 (CPPtat is SEQ ID NO: 2, and Flot-1 aa 26-41 is SEQ ID NO: 3; the full sequence is SEQ ID NO: 4).

FIG. 2A-B shows (A) flotillin-1 is overexpressed in more aggressive triple-negative breast cancer cell lines and not highly in luminal A or non-tumorigenic MCF-10A. (B) Flotillin-1 protein expression in different breast cancer cell lines and one non-cancerous mammary epithelial line. Protein expression by band intensity was normalized to GAPDH.

FIG. 3A-C shows flotillin-1 palmitoylation defect validation through click chemistry palmitoylation detection. (A) HEK293T cells were transiently transfected with flotillin-1-GFP (WT) or flotillin-1-C34A-GFP (C34A) constructs along with 25 μM of the alkyne-linked palmitate analogue (17-ODYA) before being subjected to click chemistry. The (−17-ODYA) conditions served as experimental negative controls. Cells were lysed and subjected to click chemistry with an azide-linked biotin. All palmitoylated proteins were then biotinylated and pulled down with streptavidin agarose. Palmitoylated flotillin-1-GFP was then detected by standard western blotting with a GFP antibody. (B) mRNA expression was tested between flotillin-1-GFP and flotillin-1-C34A-GFP in MDA-MB-231 breast cancer cells to ensure that there was no alteration in transcription. (C) shows a schematic for the streptavidin pulldown assay.

FIG. 4A-C shows (A) chemically blocking palmitoylation results in decreased flotillin-1 protein which is restored upon proteasomal inhibition. MDA-MB-231 and SUM 159 triple-negative breast cancer cell lines were treated with drug vehicle (DMSO), (B, C) global palmitoylation inhibitor (2-BP), or 2-BP+a proteasomal inhibitor (MG-132). Inhibition of palmitoylation significantly reduces flotillin-1 protein levels, which is restored upon proteasomal inhibition.

FIG. 5A-C shows (A) flotillin-1 knockdown (shFLOT-1+EV) reduces invasive capacity of MDA-MB-231 triple negative breast cancer cells which is rescued by re-expression of the wild type flotillin-1 (WT), but not the palmitoylation defective mutant (C34A). (B) MDA-MB-231 stable shcontrol or shFlotillin-1 cells were transfected with an empty vector (EV), flotillin-1-GFP (WT), or flotillin-1-C34A-GFP and subjected to Collagen I invasion assay using invasion chambers. The purple indicates invaded cells on the bottom of the membrane. Quantification was performed by de-staining the stained invaded cells and recording the absorbance. (C) Western blotting using the same cells in the invasion assay show that the C34A mutant is causing a decrease in flotillin-1 protein as observed with the chemical inhibition of palmitoylation with endogenous flotillin-1 (FIG. 4).

FIG. 6A-G shows that altering flotillin-1 palmitoylation status either genetically or by targeting its palmitoyl acyl transferase promotes its proteasomal dependent degradation. A) Click chemistry palmitoylation detection of wildtype (WT) or palmitoylation defective flotillin-1 protein (C34A) with or without proteasomal inhibitor (MG-132). Palm-flotillin-1 refers to palmitoylated flotillin-1 and flotillin-1 corresponds to total flotillin-1. B) Cycloheximide chase stability assay using 100 μg/ml of the protein synthesis inhibitor cycloheximide (CHX) for various time points to assess flotillin-1 protein stability. C and D) Analysis of flotillin-1 protein stability when palmitoylation status is genetically disrupted (flotillin-1-C34A). Wild type (flotillin-1) and C34A palmitoylation defective cells (flotillin-1-C34A, FIG. 6C) were subjected to protein synthesis inhibitor (CHX, FIG. 6D) with either proteasomal (MG-132) or lysosomal inhibitors CQ) to test which would rescue the protein degradation occurring in the palmitoylation defective flotillin-1 protein. E) Click chemistry palmitoylation detection of endogenous flotillin-1 protein in control or zDHHC5 (palmitoyl acyl transferase) knockdown in two triple negative breast cell lines. F) Cycloheximide protein stability assay of endogenous flotillin-1 protein in two different triple negative breast cancer cell lines. G) Rescue experiments of endogenous flotillin-1 when two different breast cancer cell lines were treated with MG-132, a proteasomal inhibitor, in the presence of the protein synthesis inhibitor (cycloheximide) in control shRNA (Con) or zDHHC5 shRNA (zD5) triple negative breast cancer cell lines.

FIG. 7A-B shows knocking down flotillin-1 protein levels does not alter apoptosis or viability in non-cancerous mammary epithelial MCF-10A cells. A) Analysis of apoptosis (cleaved PARP) in non-Cancerous mammary epithelial cell lines (MCF-10A) expressing control or flotillin-1 targeting shRNA to deplete flotillin-1 protein in these cells mimicking the flotillin-1 palmitoylation defect. B) Cell viability analysis (MTT) when non-cancerous mammary epithelial cells (MCF-10A) were depleted of flotillin-1 protein (shFLOT-1) compare to control shRNA (shCON).

FIG. 8 shows blocking flotillin-1 palmitoylation genetically causes a concomitant decrease in flotillin-2, a flotillin-1 interacting protein that also contributes to tumor progression.

FIG. 9A-F shows flotillin-1 palmitoylation defective TNBC cells display attenuated tumor growth and lung metastasis in vivo. A) Schematic representing the experimental design. dTomato expressing cells harbor flotillin-1 proteins incapable of being palmitoylated, while GFP expressing cells possess a flotillin-1 protein that is able to be palmitoylated. The cells which can be palmitoylated and those which cannot were contralaterally orthotopically implanted in the mammary fat pad of the same mice and allowed to grow and disseminate for a period of 8 weeks prior to tissue analysis of tumor burden. B) Validation staining of primary tumors for expression of flotillin-1 palmitoylation effective or flotillin-1 palmitoylation defective reporters. C) Final tumor weight after 8-weeks of growth in mice with C34A (flotillin-1 palmitoylation defective) and WT (green, palmitoylation effective). D) IHC staining of apoptosis marker (cleaved caspase-3) and proliferation (Ki67) in WT or C34A tumors. Protein expression intensity is shown. E) Metastatic burden analysis of lungs (a common site for triple negative breast cancer metastasis). There were no visible red (flotillin-1 palmitoylation defective) lung metastasis while there were multiple occurrences of green (flotillin-1 palmitoylation effective). F) Graph quantifying the number of lung metastasis.

FIG. 10A-F shows targeting flotillin-1 palmitoylation trough a peptidic competitive palmitoylation inhibitor. A) Schematic of flotillin-1 targeted palmitoylation peptide fused to a GFP tag for analysis. B) Validation that the flotillin-1 peptide sequence becoming palmitoylated. Click chemistry was used to detect palmitoylation of GFP alone (GFP-EV) or GFP fused to the flotillin-1 palmitoylation peptide sequence (GFP-F1). C) LC-MS analysis of the targeting competitive palmitoylation inhibitor (CPP-F1) chromatogram. D) shows MS spectrum analysis. E)) Sequence illustration of cell penetrating peptide sequence alone (CPP) or CPP fused to the flotilin-1 palmitoylation sequence (CPP-F1). CPP is SEQ ID NO: 2. Flotillin-1 amino acids 23-41 is from SEQ ID NO: 1. F) Analysis of flotillin-1 protein after 24 hours of in vitro treatment of two different triple negative breast cancer cells with control (CPP) or CPP-F1 at 5 and 10 μM).

### DETAILED DESCRIPTION

### Definitions

All references cited herein, including the references cited therein, are incorporated by reference in their entirety as though fully set forth. Unless defined otherwise, technical and scientific terms used herein have the same meaning as commonly understood by one of ordinary skill in the art to which this invention belongs.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

“Beneficial results” may include, but are in no way limited to, lessening or alleviating the severity of the disease condition, preventing the disease condition from worsening, curing the disease condition, preventing the disease condition from developing, lowering the chances of a patient developing the disease condition and prolonging a patient's life or life expectancy. In some embodiments, the disease condition is cancer.

“Cancer” and “cancerous” refer to or describe the physiological condition in mammals that is typically characterized by unregulated cell growth. Examples of cancer include, but are not limited to B-cell lymphomas (Hodgkin's lymphomas and/or non-Hodgkins lymphomas), brain tumor, breast cancer, colon cancer, lung cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, brain cancer, and prostate cancer.

“Chemotherapeutic drugs” or “chemotherapeutic agents” as used herein refer to drugs used to treat cancer including but not limited to Albumin-bound paclitaxel (nab-paclitaxel), Actinomycin, Alitretinoin, All-trans retinoic acid, Azacitidine, Azathioprine, Bevacizumab, Bexatotene, Bleomycin, Bortezomib, Carboplatin, Capecitabine, Cetuximab, Cisplatin, Chlorambucil, Cyclophosphamide, Cytarabine, Daunorubicin, Docetaxel, Doxifluridine, Doxorubicin, Epirubicin, Epothilone, Erlotinib, Etoposide, Fluorouracil, Gefitinib, Gemcitabine, Hydroxyurea, Idarubicin, Imatinib, Ipilimumab, Irinotecan, Mechlorethamine, Melphalan, Mercaptopurine, Methotrexate, Mitoxantrone, Ocrelizumab, Ofatumumab, Oxaliplatin, Paclitaxel, Panitumab, Pemetrexed, Rituximab, Tafluposide, Teniposide, Tioguanine, Topotecan, Tretinoin, Valrubicin, Vemurafenib, Vinblastine, Vincristine, Vindesine, Vinorelbine, Vorinostat, Romidepsin, 5-fluorouracil (5-FU), 6-mercaptopurine (6-MP), Cladribine, Clofarabine, Floxuridine, Fludarabine, Pentostatin, Mitomycin, ixabepilone, Estramustine, or a combination thereof.

“Patient outcome” refers to whether a patient survives or dies as a result of treatment. A more accurate prognosis for patients as provided in this invention increases the chances of patient survival.

“Poor prognosis” means that the prospect of survival and recovery of disease is unlikely despite the standard of care for the treatment of the cancer (for example, breast cancer), that is, surgery, radiation, chemotherapy. Poor prognosis is the category of patients whose survival is less than that of the median survival.

“Good prognosis” means that the prospect of survival and recovery of disease is likely with the standard of care for the treatment of the disease, for example, surgery, radiation, chemotherapy. Good prognosis is the category of patients whose survival is not less than that of the median survival.

A “recurrence” means that the cancer has returned after initial treatment.

“Non-recurrent” or “recurrence-free”, as used herein means that the cancer is in remission; being recurrent means that the cancer is growing and/or has metastasized, and some surgery, therapeutic intervention, and/or cancer treatment is required to lower the chance of lethality. The “non-recurrent subjects” are subjects who have non-recurrent or recurrence-free disease, and they can be used as the control for recurrent subjects who have recurrent disease or recurrence.

“Subject” or “individual” or “animal” or “patient” or “mammal,” is meant any subject, particularly a mammalian subject, for whom diagnosis, prognosis, or therapy is desired. In some embodiments, the subject has cancer. In some embodiments, the subject had cancer at some point in the subject's lifetime. In various embodiments, the subject's cancer is in remission, is recurrent or is non-recurrent.

“Mammal” as used herein refers to any member of the class Mammalia, including, without limitation, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. In certain embodiments, the mammal is a human subject. The term does not denote a particular age or sex. Thus, adult and newborn subjects, as well as fetuses, whether male or female, are intended to be included within the scope of this term.

“Therapeutic agents” as used herein refers to agents that are used to, for example, treat, inhibit, prevent, mitigate the effects of, reduce the severity of, reduce the likelihood of developing, slow the progression of and/or cure, a disease. Diseases targeted by the therapeutic agents include but are not limited to carcinomas, sarcomas, lymphomas, leukemia, germ cell tumors, blastomas, antigens expressed on various immune cells, and antigens expressed on cells associated with various hematologic diseases, autoimmune diseases, and/or inflammatory diseases.

The term “combination therapy”, as used herein, refers to those situations in which two or more different pharmaceutical agents are administered in overlapping regimens so that the subject is simultaneously exposed to both agents.

The term “palmitoylation” as used herein refers to the post-translational addition of the 16-carbon fatty acid, palmitate, to specific cysteine residues by a labile thioester linkage. In certain aspects, palmitoylation is reversible.

As used herein, the term “corresponding to” is often used to designate the position/identity of an amino acid residue in a polypeptide. Those of ordinary skill will appreciate that, for purposes of simplicity, a canonical numbering system is typically used when referring to positions in a polypeptide chain, so that an amino acid “corresponding to” a residue at position 190, for example, need not actually be the 190th amino acid in a particular amino acid chain but rather corresponds to the residue found at 190 in a reference polypeptide (e.g., a wild type polypeptide); those of ordinary skill in the art readily appreciate how to identify corresponding amino acids.

The term “direct” may be used herein to refer to a physical interaction between two entities. Typically, a “direct” interaction is a non-covalent interaction that does not require intermediating entities. In some embodiments, a direct interaction is one that occurs in the absence of one or more other entities (e.g., of entities not participating in the interaction and/or in its detection). In some embodiments, a direct interaction is one that occurs in the absence of any other entities.

As used herein, the term “identity” refers to the overall relatedness between polymeric molecules, e.g., between nucleic acid molecules (e.g., DNA molecules and/or RNA molecules) and/or between polypeptide molecules. Calculation of the percent identity of two nucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

In general, an agent is said to “inhibit” a target if level and/or activity of the target is reduced in a system producing and/or containing the target when the agent is present as compared to otherwise identical conditions when it is absent. In some embodiments, level and/or activity of the target is reduced at least 5%, 10%, 15%, 20%, 25%, 30%, 35%, 40%, 45%, 50%, 55%, 60%, 65%, 70%, 75%, 80%, 85%, 90%, 95%, 100% or more when the agent is present; in some embodiments, level and/or activity of the target is reduced at least 1.5 fold, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, 150 fold, 200 fold, 250 fold, 300 fold, 350 fold, 400 fold, 450 fold, 500 fold, 550 fold, 600 fold, 650 fold, 700 fold, 750 fold, 800 fold, 850 fold, 900 fold, 950 fold, 1000 fold or more when the agent is present as compared with when it is absent.

The term “isolated”, as used herein, refers to an agent or entity that has either (i) been separated from at least some of the components with which it was associated when initially produced (whether in nature or in an experimental setting); or (ii) produced by the hand of man. Isolated agents or entities may be separated from at least about 10%, 20%, 30%, 40%, 50%, 60%, 70%, 80%, 90%, or more of the other components with which they were initially associated. In some embodiments, isolated agents are more than 90%, 91%, 92%, 93%, 94%, 95%, 96%, 97%, 98%, 99% pure. In some embodiments, calculation of percent purity of isolated substances and/or entities does not include excipients (e.g., buffer, solvent, water, etc.) Non-natural amino acid:

A “polypeptide”, generally speaking, is a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides sometimes include “non-natural” amino acids or other entities that nonetheless are capable of integrating into a polypeptide chain, optionally. Those of ordinary skill in the art will further appreciate that particular classes of polypeptides can be defined based on a designated degree of structural and/or functional similarity. In general, polypeptides of a particular class may be defined as having a specified degree of overall sequence identity (e.g., at least 30%, 31%, 32%, 33%, 34%, 35%, 36%, 37%, 38%, 39%0, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%<87%, 88%, 89%, 90%, 91%, 92%, 93%, 94%, 95%, 96%, 97%, 98%, 99%, or more) and/or as sharing one or more characteristic sequence elements. In some embodiments, such a characteristic sequence element is one whose presence correlates with a particular biological activity.

As used herein, an agent or entity is “pure” if it is substantially free of other components. For example, a preparation that contains more than about 90% of a particular agent or entity is typically considered to be a pure preparation. In some embodiments, an agent or entity is at least 91%, 92%, 93%, 94%, 95%, 96%, 97%, 98%, or 99% pure.

The term “flotillin-1 palmitoylation modulator” is used herein to refer to agents for which the level and/or activity of palmitoylated flotillin-1 is altered when the agent is present than under otherwise identical conditions lacking the agent. Level and/or activity of palmitoylated flotillin-1 may be assessed according to any appropriate method including, for example, those described herein. In some embodiments, level and/or activity of palmitoylated flotillin-1 is at least 5%, 10%, 15%, 20%, 25%, 30%, 35%, 40%, 45%, 50%, 55%, 60%, 65%, 70%, 75%, 80%, 85%, 90%, 95%, 100% or more different when the agent is present than under otherwise identical conditions when it is absent. In some embodiments, level and/or activity of palmitoylated flotillin-1 is at least 1.5 fold, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, 150 fold, 200 fold, 250 fold, 300 fold, 350 fold, 400 fold, 450 fold, 500 fold, 550 fold, 600 fold, 650 fold, 700 fold, 750 fold, 800 fold, 850 fold, 900 fold, 950 fold, 1000 fold or more different when the agent is present than under otherwise identical conditions when it is absent. In some embodiments, a flotillin-1 palmitoylation modulator is a flotillin-1 palmitoylation inhibitor. In some embodiments, a flotillin-1 palmitoylation modulator interacts directly with an enzyme that palmitoylates flotillin-1 (e.g., with a flotillin-1 palmitoyl-acyl transferase). In some embodiments, a flotillin-1 palmitoylation modulator interacts directly with an enzyme that participates in production of palmitate; in some such embodiments, a flotillin-1 palmitoylation modulator interacts directly with a fatty acid synthase.

The term “flotillin-1 palmitoylation inhibitor” is used herein to refer to any agent for which the level and/or activity of palmitoylated flotillin-1 is lower when the agent is present than under otherwise identical conditions lacking the agent. Level and/or activity of palmitoylated flotillin-1 may be assessed according to any appropriate method including, for example, those described herein. In some embodiments, level and/or activity of palmitoylated flotillin-1 is at least 5%, 10%, 15%, 20%, 25%, 30%, 35%, 40%, 45%, 50%, 55%, 60%, 65%, 70%, 75%, 80%, 85%, 90%, 95%, 100% or more lower when the agent is present than under otherwise identical conditions when it is absent. In some embodiments, level and/or activity of palmitoylated RAS is at least 1.5 fold, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, 150 fold, 200 fold, 250 fold, 300 fold, 350 fold, 400 fold, 450 fold, 500 fold, 550 fold, 600 fold, 650 fold, 700 fold, 750 fold, 800 fold, 850 fold, 900 fold, 950 fold, 1000 fold or more lower when the agent is present than under otherwise identical conditions when it is absent. In some embodiments, a flotillin-1 palmitoylation inhibitor acts on (in some embodiments directly; in some embodiments indirectly) a flotillin-1 palmitoyl-acetyl transferase. In some embodiments, a flotillin-1 palmitoylation inhibitor acts (in some embodiments directly; in some embodiments indirectly) on a fatty acid synthase, for example on a fatty acid synthase whose activity results in production of palmitate.

The term “RNAi-inducing agent” is used to refer to siRNAs, shRNAs, and other double-stranded structures (e.g., dsRNA) that can be processed to yield an siRNA or shRNA or other small RNA species that inhibits expression of a target transcript by RNA interference. In certain embodiments of the invention an RNAi-inducing agent inhibits expression of a target RNA via an RNA interference pathway that involves translational repression.

The term “RNAi-inducing entity”, encompasses RNA molecules and vectors whose presence within a cell results in RNAi and leads to reduced expression of a transcript to which the RNAi-inducing entity is targeted. The RNAi-inducing entity may be, for example, an RNAi-inducing agent such as an siRNA, shRNA, or an RNAi-inducing vector. Use of the terms “RNAi-inducing entity”, “RNAi-inducing agent”, or “RNAi-inducing vector” is not intended to imply that the entity, agent, or vector upregulates or activates RNAi in general, though it may do so, but simply to indicate that presence of the entity, agent, or vector within the cell results in RNAi-mediated reduction in expression of a target transcript. An “RNAi-inducing entity” as used herein is an entity that has been modified or generated by the hand of man and/or whose presence in a cell is a result of human intervention as distinct, e.g., from endogenous RNA species or RNA species that are produced in a cell during the natural course of viral infection.

An “RNAi-inducing vector” is a vector whose presence within a cell results in transcription of one or more RNAs that hybridize to each other or self-hybridize to form an RNAi-inducing agent such as an siRNA or shRNA. In various embodiments of the invention this term encompasses plasmids or viruses whose presence within a cell results in production of one or more RNAs that self-hybridize or hybridize to each other to form an RNAi-inducing agent. In general, the vector comprises a nucleic acid operably linked to expression signal(s) so that one or more RNA molecules that hybridize or self-hybridize to form an RNAi-inducing agent is transcribed when the vector is present in a cell. Thus the vector provides a template for intracellular synthesis of the RNAi-inducing agent. For purposes of inducing RNAi, presence of a viral genome in a cell constitutes presence of the virus within the cell. A vector is considered to be present within a cell if it is introduced into the cell, enters the cell, or is inherited from a parental cell, regardless of whether it is subsequently modified or processed within the cell. An RNAi-inducing vector is considered to be targeted to a transcript if the vector comprises a template for transcription of an RNAi-inducing agent that is targeted to the transcript. Such vectors have a number of other uses in addition to transcript inhibition in a cell. For example, they may be used for in vitro production of an RNAi-inducing agent and/or for production of the agent in a cell that may or may not contain a transcript to which the vector is targeted.

A “short, interfering RNA” comprises a double-stranded (duplex) RNA that is between 15 and approximately 29 nucleotides in length or any other subrange or specific value within the interval between 15 and 29, e.g., 16-18, 17-19, 21-23, 24-27, 27-29 nt long and optionally further comprises one or two single-stranded overhangs, e.g., a 3′ overhang on one or both strands. In certain embodiments the duplex is approximately 19 nt long. The overhang may be, e.g., 1-6 residues in length, e.g., 2 nt. An siRNA may be formed from two RNA molecules that hybridize together or may alternatively be generated from an shRNA. In certain embodiments of the invention one or both of the 5′ ends of an siRNA has a phosphate group while in other embodiments one or more of the 5′ ends lacks a phosphate group. In certain embodiments of the invention one or both of the 3′ ends has a hydroxyl group while in other embodiments they do not. One strand of an siRNA, which is referred to as the “antisense strand” or “guide strand” includes a portion that hybridizes with a target transcript. In certain preferred embodiments of the invention, the antisense strand of the siRNA is 100% complementary with a region of the target transcript, i.e., it hybridizes to the target transcript without a single mismatch or bulge over a target region between 15 and approximately 29 nt in length, preferably at least 16 nt in length, more preferably 18-20, e.g., 19 nt in length. The region of complementarity may be any subrange or specific value within the interval between 17 and 29, e.g., 17-18, 19-21, 21-23, 19-23, 24-27, 27-29. In other embodiments the antisense strand is substantially complementary to the target region, i.e., one or more mismatches and/or bulges exists in the duplex formed by the antisense strand and a target transcript. The two strands of an siRNA are substantially complementary, preferably 100% complementary to each other within the duplex portion.

The term “short hairpin RNA” refers to an RNA molecule comprising at least two complementary portions hybridized or capable of hybridizing to form a double-stranded (duplex) structure sufficiently long to mediate RNAi (as described for siRNA duplexes), and at least one single-stranded portion that forms a loop connecting the regions of the shRNA that form the duplex. The structure is also referred to as a stem/loop structure, with the stem being the duplex portion. The structure may further comprise an overhang (e.g., as described for siRNA) on the 5′ or 3′ end. Preferably, the loop is about 1-20, more preferably about 4-10, and most preferably about 6-9 nt long and/or the overhang is about 1-20, and more preferably about 2-15 nt long. The loop may be located at either the 5′ or 3′ end of the region that is complementary to the target transcript whose inhibition is desired (i.e., the antisense portion of the shRNA). In certain embodiments the overhang comprises one or more U residues, e.g., between 1 and 5 Us. As described further below, shRNAs are processed into siRNAs by the conserved cellular RNAi machinery. Thus shRNAs are precursors of siRNAs and are, in general, similarly capable of inhibiting expression of a target transcript that is complementary to a portion of the shRNA (referred to as the antisense or guide strand of the shRNA). In general, the features of the duplex formed between the antisense strand of the shRNA and a target transcript are similar to those of the duplex formed between the guide strand of an siRNA and a target transcript. In certain embodiments of the invention the 5′ end of an shRNA has a phosphate group while in other embodiments it does not. In certain embodiments of the invention the 3′ end of an shRNA has a hydroxyl group while in other embodiments it does not.

As is known in the art, “specificity” is a measure of the ability of a particular ligand or agent to distinguish its binding and/or reaction partner from other potential binding and/or reaction partners in its environment. In some embodiments, a ligand or agent is considered to show “specificity” for its binding and/or reaction partner if it shows at least a 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 125, 150, 175, 200, 225, 250, 275, 300, 325, 350, 375, 400, 425, 450, 475, 500, 550, 600, 650, 700, 750, 800, 850, 900, 950, 1000, 2000, 3000, 4000, 5000, 6000, 7000, 8000, 9000, 10000 fold preference or more for its binding and/or reaction partner over other potential binding and/or reaction partners in its environment.

As used herein, the phrase “therapeutic agent” refers to any agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of and/or reduce incidence of one or more symptoms or features of influenza infection.

As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

The expression “unit dose” as used herein refers to a physically discrete unit of a formulation appropriate for a subject to be treated. It will be understood, however, that the total daily usage of a formulation of the present invention will be decided by the attending physician within the scope of sound medical judgment. The specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts. A particular unit dose may or may not contain a therapeutically effective amount of a therapeutic agent.

As used herein, the term “variant” is a relative term that describes the relationship between a particular polypeptide of interest and a reference polypeptide to which its sequence is being compared. A polypeptide of interest is considered to be a “variant” of a reference polypeptide if the polypeptide of interest has an amino acid sequence that is identical to that of the reference but for a small number of sequence alterations at particular positions. Typically, fewer than 20%, 15%, 10%, 9%, 8%, 7%, 6%, 5%, 4%, 3%, 2% of the residues in the variant are substituted as compared with the reference. In some embodiments, a variant has 10, 9, 8, 7, 6, 5, 4, 3, 2, or 1 substituted residue as compared with a reference. Often, a variant has a very small number (e.g., fewer than 5, 4, 3, 2, or 1) number of substituted functional residues (i.e., residues that participate in a particular biological activity). Furthermore, a variant typically has not more than 5, 4, 3, 2, or 1 additions or deletions, and often has no additions or deletions, as compared with the reference. Moreover, any additions or deletions are typically fewer than about 25, 20, 19, 18, 17, 16, 15, 14, 13, 10, 9, 8, 7, 6, and commonly are fewer than about 5, 4, 3, or 2 residues. In some embodiments, the reference polypeptide is one found in nature.

As used herein, “vector” refers to a nucleic acid molecule capable of transporting another nucleic acid to which it has been linked. In some embodiment, vectors are capable of extra-chromosomal replication and/or expression of nucleic acids to which they are linked in a host cell such as a eukaryotic or prokaryotic cell. Vectors capable of directing the expression of operatively linked genes are referred to herein as “expression vectors.”

As is understood in the art, the phrase “wild type” generally refers to a normal form of a protein or nucleic acid, as is found in nature.

As used herein, the term “cancer treatment” means any treatment for cancer known in the art including, but not limited to, chemotherapy and radiation therapy.

As used herein, “tumor cells” means both cells derived from tumors, including malignant tumors, and cells immortalized in vitro. “Normal” cells refer to cells with normal growth characteristics that do not show abnormal proliferation.

As used herein, the terms “an individual identified as having cancer” and “cancer patient” are used interchangeably and are meant to refer to an individual who has been diagnosed as having cancer. There are numerous well known means for identifying an individual who has cancer. In some embodiments, a cancer diagnosis is made or confirmed using PET imaging. Some embodiments of the present disclosure comprise the step of identifying individuals who have cancer.

As used herein, the term “therapeutically effective amount” is meant to refer to an amount of an active agent or combination of agents effective to ameliorate or prevent the symptoms, shrink tumor size, or prolong the survival of the patient being treated. Determination of a therapeutically effective amount is well within the capabilities of those skilled in the art, especially in light of the detailed disclosure provided herein.

As used herein the term “inhibit” or “inhibiting” refers to a statistically significant and measurable reduction in activity, preferably a reduction of at least about 10% versus control, more preferably a reduction of about 50% or more, still more preferably a reduction of about 80% or more.

As used herein the term “increase” or “enhancing” refers to a statistically significant and measurable increase in activity, preferably an increase of at least about 10% versus control, more preferably an increase of about 50% or more, still more preferably an increase of about 80% or more.

The term “prevent” or “preventing” when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Preventing metastasis means preventing the spread of cancer beyond the initial site of the tumor or cancer cells.

The terms “treatment”, “treating”, “treat”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease and/or relieving one or more disease symptoms. “Treatment” is also meant to encompass delivery of an agent in order to provide for a pharmacologic effect, even in the absence of a disease or condition. For example, “treatment” encompasses delivery of a palmitoylation modulator that can provide for enhanced or desirable effects in the subject (e.g., reduction of metastatic rate, etc.).

### General Description

The gene for flotillin-1 was initially cloned by Bickel et al. (Journal of Biological Chemistry 1997, 272:13793-13802; the contents of which are incorporated herein by reference) in an attempt to identify genes for novel proteins that are enriched in the membranes of purified caveolae from murine lung tissue. The cDNA for murine flotillin-1 encodes a protein of 428 amino acids with a predicted molecular weight of 47 kDa. The human flotillin-1 gene sequence (Genbank Accession No. AF 089750) was deposited in the Genbank data base by A. J. Edgar (Imperial College, London, UK) by direct submission in 1998. The human flotillin-1 gene encodes a protein of 427 amino acids. The amino acid sequences of the human and murine flotillin-1 protein are highly conserved (>98%). The flotillin-1 gene displays significant homology to ESA (epidermal surface antigen, also named flotillin-2). Although flotillin-1 and ESA have different N-termini, they share a region of 47% identity on the amino acid level. Furthermore, there is a modest (24%) homology to two hypothetical open reading frames from the cyanobacterium Synechococcus. Flotillin-1 is expressed in most tissues.

Flotillin-1 has been shown to play a role in cancer (Liu XX, Liu WD, Wang L, et al. Roles of flotillins in tumors. J Zhejiang Univ Sci B. 2018; 19(3):171-182. doi:10.1631/jzus.B1700102, which is herein incorporated by reference), particularly in cancer metastasis. Expression of flotillin-1 has been associated with metastasis in several solid tumors. However, the relatedness of palmitoylation of flotillin-1 and cancer was not understood until now. Disclosed herein are methods and compositions which reduce, inhibit, or prevent flotillin-1's role in cancer metastasis by decreasing palmitoylation of flotillin-1.

As mentioned above, the methods disclosed herein of modulating palmitoylation of flotillin-1 can be used to reduce, inhibit, or prevent cancer metastasis. By “reduce” or “inhibit” is meant that metastasis of cancer in an individual is reduced or inhibited by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, as compared to a control, or to a subject before treatment with a composition which decreases palmitoylation of flotillin-1.

By “decreases palmitoylation of flotillin-1” is meant that palmitoylation of flotillin-1 is decreased at one or more sites in the flotillin-1 protein by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%. It is noted that palmitoylation can occur at multiple sites within flotillin-1, and one or more of these sites can have palmitoylation reduced or inhibited. Palmitoylation of flotillin-1 can be temporarily decreased, so that it is reversible upon removal of the composition which decreases palmitoylation of flotillin-1, or can be permanent, or irreversible.

It is noted that decreasing palmitoylation of flotillin-1 can have a number of effects on flotillin-1. For example, decreasing palmitoylation can modulate the stability of flotillin-1. When this occurs, stability of flotillin-1 can be decreased by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, for example.

Decreasing palmitoylation of flotillin-1 can also modulate intermolecular interactions of flotillin-1. When this occurs, intermolecular interactions of flotillin-1 can be decreased by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, for example flotillin-1 palmitoylation has been demonstrated to contribute to its ability to transport the insulin-like growth factor receptor (IGF-1R) from the endoplasmic reticulum to the plasma membrane and physically interacts with the receptor.

Decreasing palmitoylation of flotillin-1 can also modulate post-translational modifications of other proteins by flotillin-1. When this occurs, post-translational modifications of other proteins by flotillin-1 can be decreased by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, for example flotillin-1 has not been shown to directly post-translationally modify proteins, since it does not contain any enzymatic activity, but rather leads receptor stabilization through the lyso-endosomal system and/or facilitate plasma membrane localization. Receptors involved in cancer invasion and metastasis affected by flotillin-1 include epidermal growth factor receptor (EGFR), human epidermal growth factor receptor-2 (HER-2), Axl, Insulin-like Growth factor receptor (IGF-1R) and Transforming Growth Factor-Beta Receptor (TGF-βR). By promoting the localization, plasma membrane organization through lipid rafts, and endocytosis, flotillin-1 drives the stability of these oncogenic receptors and prolongs the phosphorylation status which leads to enhanced post-translational modification of their downstream targets ultimately contributing to cancer invasive and metastatic programming.

Modulation of palmitoylation of flotillin-1 can decrease cancer metastasis in a subject. As such, the disclosed methods and compositions can be used to prevent, abate, minimize, control, and/or lessen cancer metastasis in humans and animals. The disclosed compositions and methods can also be used to slow the rate of primary tumor growth. The disclosed compositions and methods, when administered to a subject in need of treatment, can be used to stop the spread of cancer cells. This decrease of cancer metastasis, slowing of primary tumor growth, or spreading of cancer cells, can decrease tumor growth or spread or metastasis by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, for example decreasing total flotillin-1 protein levels in tumors by short hairpin RNA(shRNA), small interfering RNA (siRNA), or by genetically removing the locus (CRISPR) attenuates invasion of the primary tumor and subsequent metastasis in multiple solid tumors.

As such, the methods and compositions disclosed herein can be administered as part of a combination therapy with one or more drugs or other pharmaceutical agents. When used as part of the combination therapy, the decrease in metastasis and reduction in primary tumor growth afforded by the disclosed methods and compositions for a more effective and efficient use of any pharmaceutical or drug therapy being used to treat the patient. In addition, control of metastasis by the disclosed compound affords the subject a greater ability to concentrate the disease in one location.

Disclosed herein are methods of treating or preventing cancer, the method comprising decreasing palmitoylation of flotillin-1 in a subject with, or at risk of developing, cancer. Relatedly, disclosed herein are methods for preventing metastasis of malignant tumors or other cancerous cells as well as to reduce the rate of tumor growth. The methods comprise administering an effective amount of one or more of the disclosed compositions, or carrying out one or more of the disclosed methods, to a subject diagnosed with a malignant tumor or cancerous cells or to a subject having a tumor or cancerous cells.

Further disclosed herein is the use of the disclosed methods and compositions for making a medicament for preventing metastasis of malignant tumors or other cancerous cells and for slowing tumor growth.

The following are non-limiting examples of cancers that can be treated by the disclosed methods and compositions: Acute Lymphoblastic; Acute Myeloid Leukemia; Adrenocortical Carcinoma; Adrenocortical Carcinoma, Childhood; Appendix Cancer; Basal Cell Carcinoma; Bile Duct Cancer, Extrahepatic; Bladder Cancer; Bone Cancer; Osteosarcoma and Malignant Fibrous Histiocytoma; Brain Stem Glioma, Childhood; Brain Tumor, Adult; Brain Tumor, Brain Stem Glioma, Childhood; Brain Tumor, Central Nervous System Atypical Teratoid/Rhabdoid Tumor, Childhood; Central Nervous System Embryonal Tumors; Cerebellar Astrocytoma; Cerebral Astrocytoma/Malignant Glioma; Craniopharyngioma; Ependymoblastoma; Ependymoma; Medulloblastoma; Medulloepithelioma; Pineal Parenchymal Tumors of Intermediate Differentiation; Supratentorial Primitive Neuroectodermal Tumors and Pineoblastoma; Visual Pathway and Hypothalamic Glioma; Brain and Spinal Cord Tumors; Breast Cancer; Bronchial Tumors; Burkitt Lymphoma; Carcinoid Tumor; Carcinoid Tumor, Gastrointestinal; Central Nervous System Atypical Teratoid/Rhabdoid Tumor; Central Nervous System Embryonal Tumors; Central Nervous System Lymphoma; Cerebellar Astrocytoma; Cerebral Astrocytoma/Malignant Glioma, Childhood; Cervical Cancer; Chordoma, Childhood; Chronic Lymphocytic Leukemia; Chronic Myelogenous Leukemia; Chronic Myeloproliferative Disorders; Colon Cancer; Colorectal Cancer; Craniopharyngioma; Cutaneous T-Cell Lymphoma; Esophageal Cancer; Ewing Family of Tumors; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastrointestinal Carcinoid Tumor; Gastrointestinal Stromal Tumor (GIST); Germ Cell Tumor, Extracranial; Germ Cell Tumor, Extragonadal; Germ Cell Tumor, Ovarian; Gestational Trophoblastic Tumor; Glioma; Glioma, Childhood Brain Stem; Glioma, Childhood Cerebral Astrocytoma; Glioma, Childhood Visual Pathway and Hypothalamic; Hairy Cell Leukemia; Head and Neck Cancer; Hepatocellular (Liver) Cancer; Histiocytosis, Langerhans Cell; Hodgkin Lymphoma; Hypopharyngeal Cancer; Hypothalamic and Visual Pathway Glioma; Intraocular Melanoma; Islet Cell Tumors; Kidney (Renal Cell) Cancer; Langerhans Cell Histiocytosis; Laryngeal Cancer; Leukemia, Acute Lymphoblastic; Leukemia, Acute Myeloid; Leukemia, Chronic Lymphocytic; Leukemia, Chronic Myelogenous; Leukemia, Hairy Cell; Lip and Oral Cavity Cancer; Liver Cancer; Lung Cancer, Non-Small Cell; Lung Cancer, Small Cell; Lymphoma, AIDS-Related; Lymphoma, Burkitt; Lymphoma, Cutaneous T-Cell; Lymphoma, Hodgkin; Lymphoma, Non-Hodgkin; Lymphoma, Primary Central Nervous System; Macroglobulinemia, Waldenstrom; Malignant Fibrous Histiocytoma of Bone and Osteosarcoma; Medulloblastoma; Melanoma; Melanoma, Intraocular (Eye); Merkel Cell Carcinoma; Mesothelioma; Metastatic Squamous Neck Cancer with Occult Primary; Mouth Cancer; Multiple Endocrine Neoplasia Syndrome, (Childhood); Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple; Myeloproliferative Disorders, Chronic; Nasal Cavity and Paranasal Sinus Cancer; Nasopharyngeal Cancer; Neuroblastoma; Non-Small Cell Lung Cancer; Oral Cancer; Oral Cavity Cancer; Oropharyngeal Cancer; Osteosarcoma and Malignant Fibrous Histiocytoma of Bone; Ovarian Cancer; Ovarian Epithelial Cancer; Ovarian Germ Cell Tumor; Ovarian Low Malignant Potential Tumor; Pancreatic Cancer; Pancreatic Cancer, Islet Cell Tumors; Papillomatosis; Parathyroid Cancer; Penile Cancer; Pharyngeal Cancer; Pheochromocytoma; Pineal Parenchymal Tumors of Intermediate Differentiation; Pineoblastoma and Supratentorial Primitive Neuroectodermal Tumors; Pituitary Tumor; Plasma Cell Neoplasm/Multiple Myeloma; Pleuropulmonary Blastoma; Primary Central Nervous System Lymphoma; Prostate Cancer; Rectal Cancer; Renal Cell (Kidney) Cancer; Renal Pelvis and Ureter, Transitional Cell Cancer; Respiratory Tract Carcinoma Involving the NUT Gene on Chromosome 15; Retinoblastoma; Rhabdomyosarcoma; Salivary Gland Cancer; Sarcoma, Ewing Family of Tumors; Sarcoma, Kaposi; Sarcoma, Soft Tissue; Sarcoma, Uterine; Sezary Syndrome; Skin Cancer (Nonmelanoma); Skin Cancer (Melanoma); Skin Carcinoma, Merkel Cell; Small Cell Lung Cancer; Small Intestine Cancer; Soft Tissue Sarcoma; Squamous Cell Carcinoma, Squamous Neck Cancer with Occult Primary, Metastatic; Stomach (Gastric) Cancer; Supratentorial Primitive Neuroectodermal Tumors; T-Cell Lymphoma, Cutaneous; Testicular Cancer; Throat Cancer; Thymoma and Thymic Carcinoma; Thyroid Cancer; Transitional Cell Cancer of the Renal Pelvis and Ureter; Trophoblastic Tumor, Gestational; Urethral Cancer; Uterine Cancer, Endometrial; Uterine Sarcoma; Vaginal Cancer; Vulvar Cancer; Waldenstrom Macroglobulinemia; and Wilms Tumor.

In one example, palmitoylation of flotillin-1 is decreased by providing a peptide inhibitor of flotillin-1. For instance, a competitive inhibitor of flotillin-1 can be used. This competitive inhibitor can, for example, comprise a cell penetrating peptide. An example of the entire flotillin-1 protein is represented by SEQ ID NO: 1. One of skill in the art will appreciate that this is a representation of flotillin-1, but one or more mutations can occur in SEQ ID NO: 1, and the sequence would still function as flotillin-1 and can still be considered representative of a functional flotillin-1 protein.

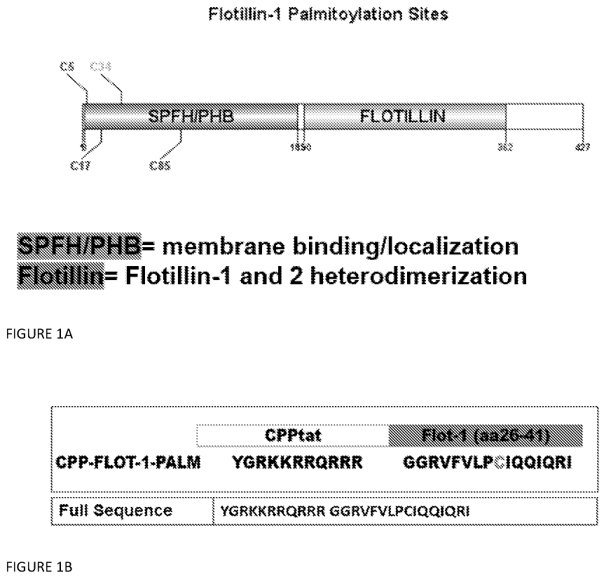

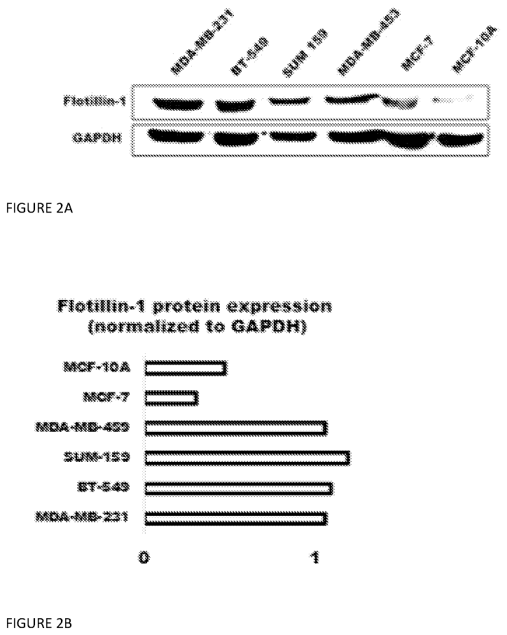

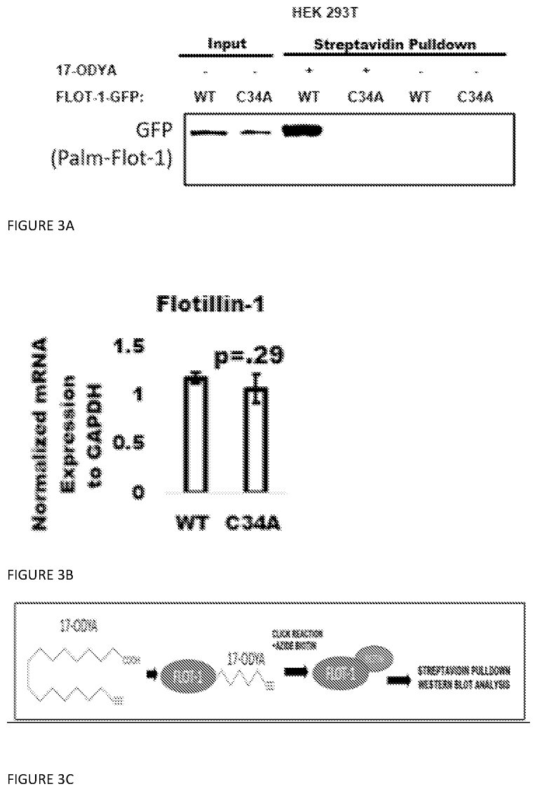

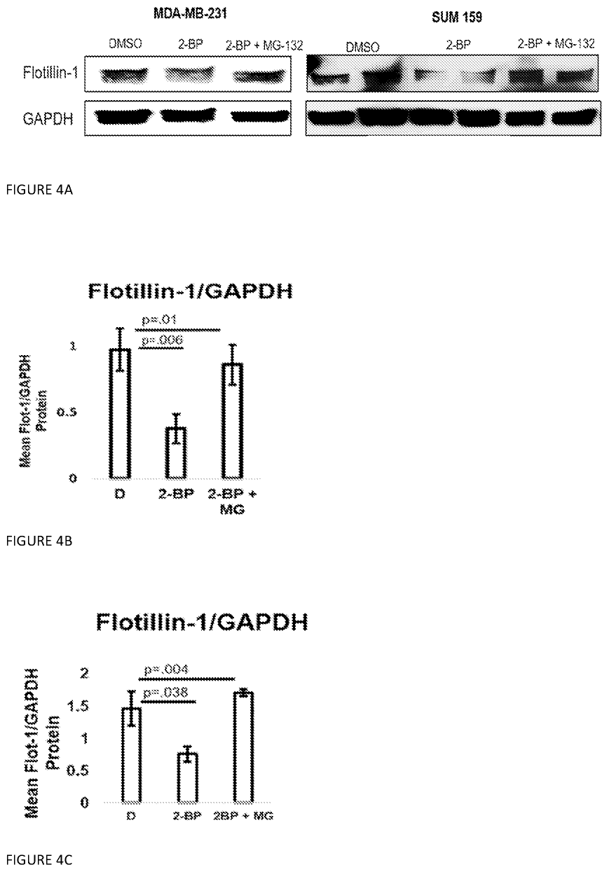

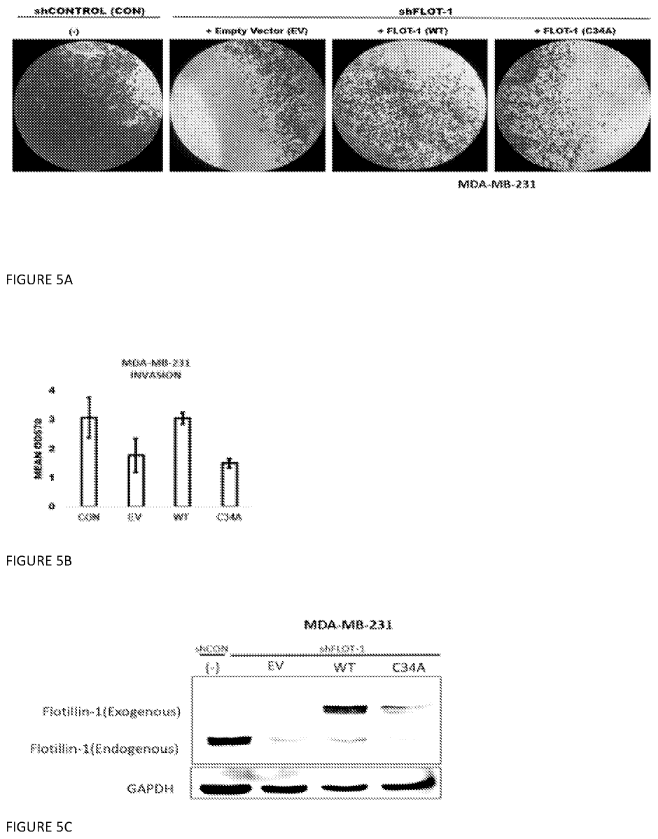

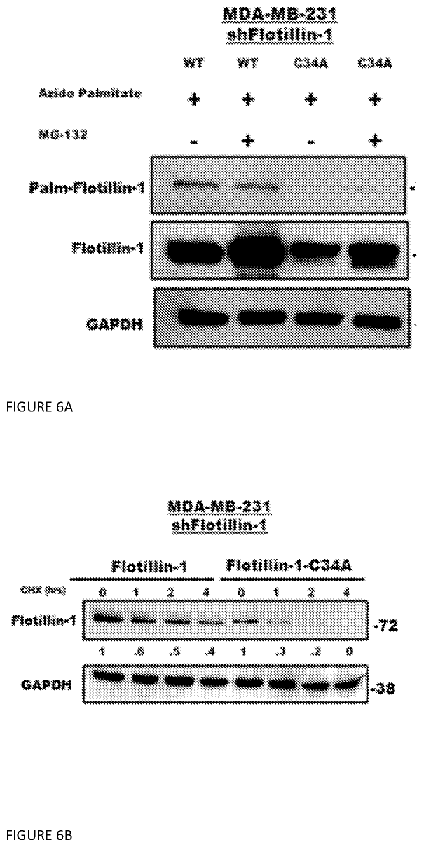

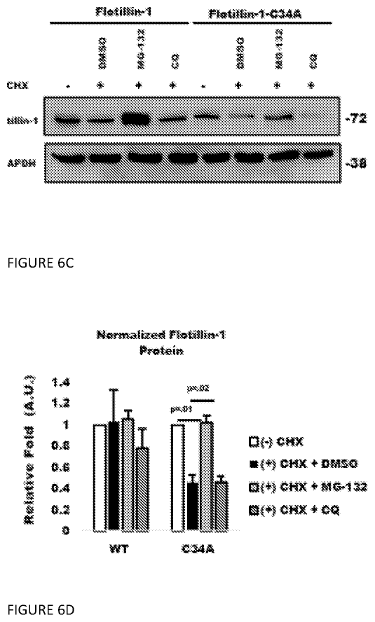

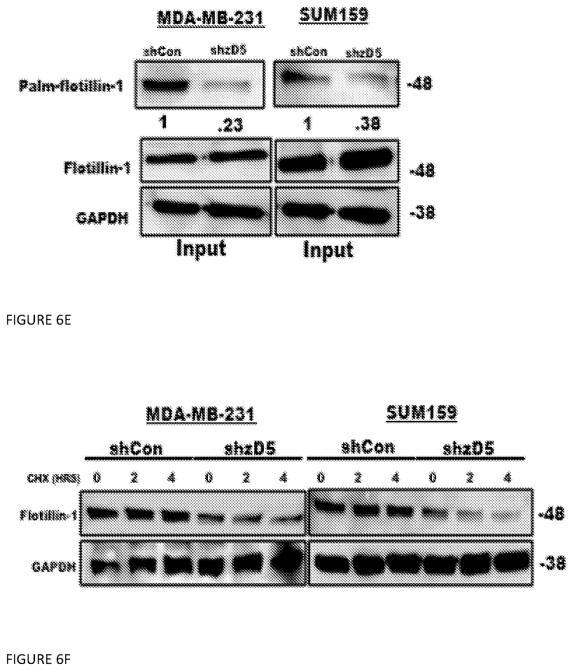

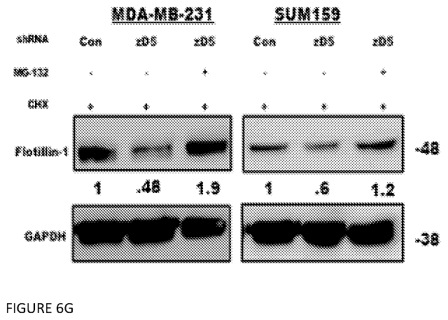

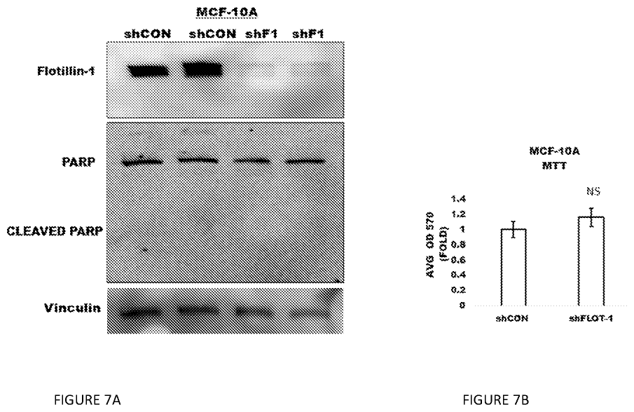

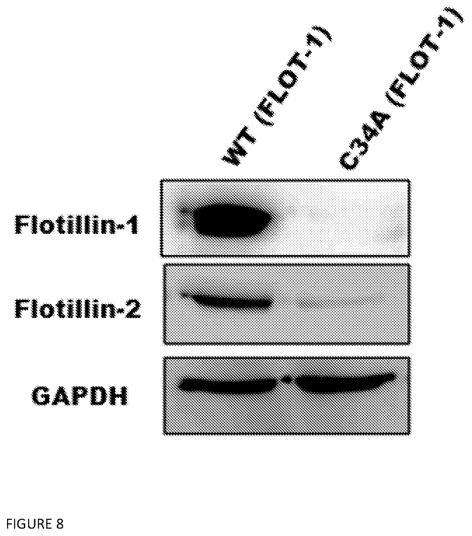

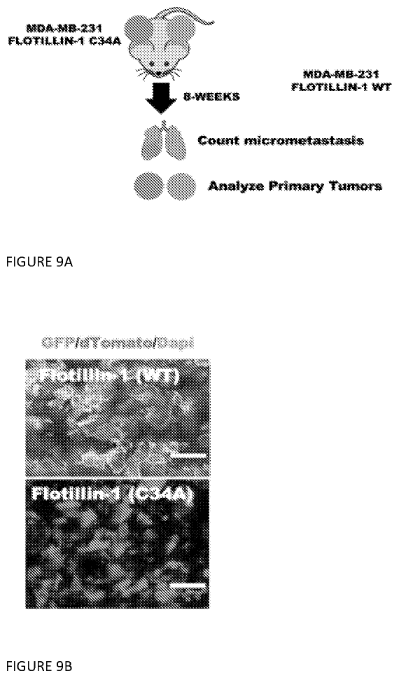

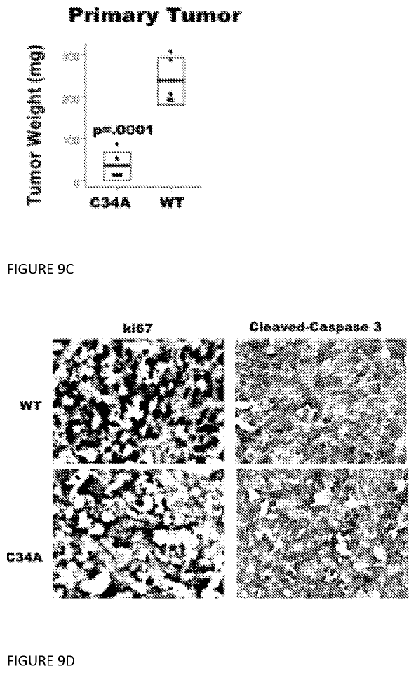

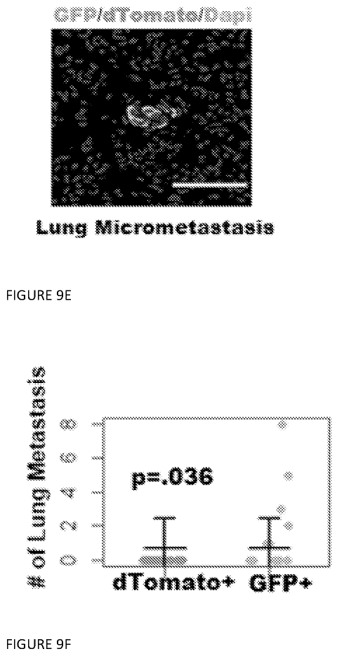

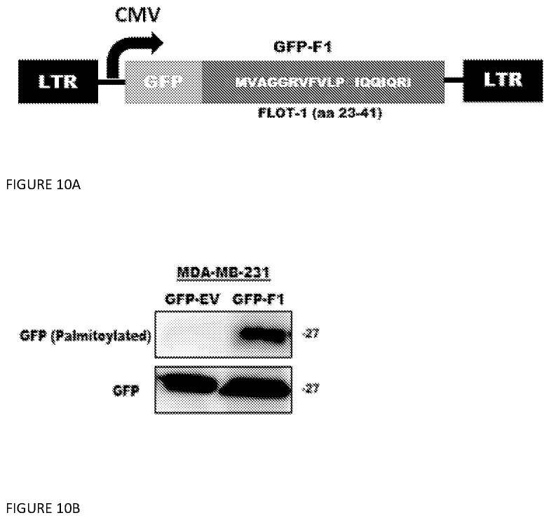

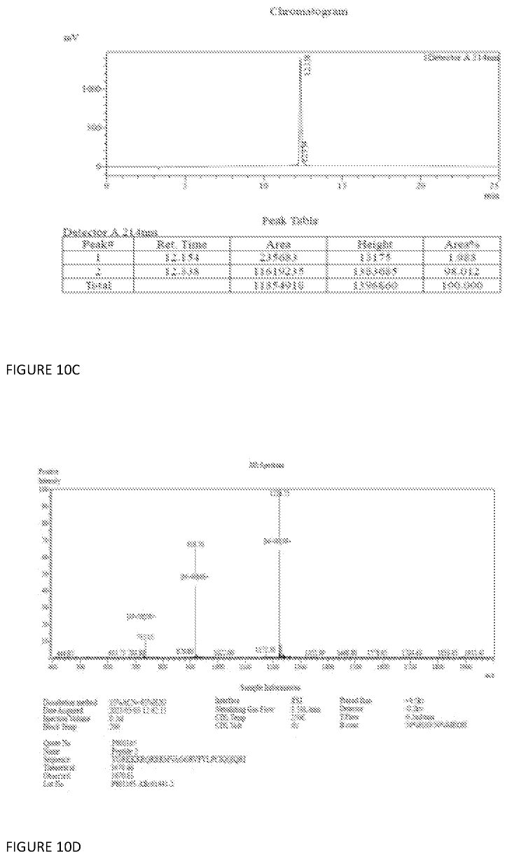

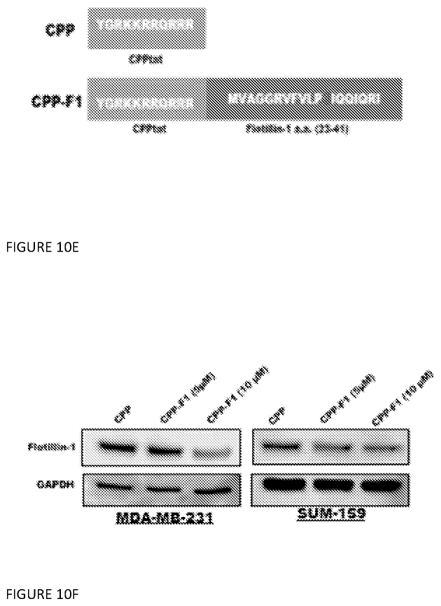

Flotillin-1 is known to possess a number of sites of palmitoylation. For example, as shown in FIG. 1, these sites can exist at positions C5, C17, C34, and C85 of SEQ ID NO: 1. Other palmitoylation sites can also exist, but these four serve as representatives. A competitive inhibitor, or decoy, can make use of one or more of palmitoylation sites, such that the decoy molecule is palmitoylated instead of the actual flotillin-1, thereby decreasing the percentage of actual flotillin-1 proteins which are palmitoylated. This can lead to a reduction of palmitoylated full-length flotillin-1 molecules, which can lead to a reduction in tumor growth, cancer metastasis, etc., as discussed above.

The competitive inhibitor of flotillin-1 can be any length, and can include at least one palmitoylation site including C5, C17, C34, and C85. For example, the competitive inhibitor (also referred to herein as the decoy) can comprise one of C5, C17, C34, and C85. Alternatively, the competitor can include two palmitoylation sites, such as C5 and C17, C5 and C34, C5 and C85, C17 and C34, C17 and C85, or C34 and C85. In another example, the competitor can include three palmitoylation sites, such as C5, C17, and C34, or C17, C34, and C85. In another example, the competitor can include all four palmitoylation sites, including C5, C17, C34, and C85. The competitor can optionally include other palmitoylation sites which are not enumerated here. It is noted that these examples are intended as examples only, and one of skill in the art can easily envision various combinations and permutations of these palmitoylation sites.

The flotillin-1 portion of the competitive inhibitor can be any length which is capable of eliciting palmitoylation, and can, for example, be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 89, 80, 81, 82, 83, 84, 85, or more amino acids in length. One of skill in the art can readily assess what an appropriate peptide length for the decoy molecule is, and can design it accordingly.

For example, the decoy, or competitive inhibitor, can include a cell penetrating peptide (CPP) or other modification which allows for cell penetration or other specific cell-targeting mechanisms. Known CPP sequences used in both clinical trials and in vivo studies to deliver both targeting peptides, proteins, siRNAs, nanoparticles, and other therapeutics into cells include TAT: GRKKRRQRRRPQ (SEQ ID NO: 11), Penetratin: RQIKIWFQNRRMKWKK (SEQ ID NO: 12), Pep-1: KETWWETWWTEWSQP-KKKRKV (SEQ ID NO: 13), MPG: GALFLGFLGAAGSTMGAWSQP-KKKRKV (SEQ ID NO: 14), and Polyarginine (R9, R8): RRRRRRRRR (SEQ ID NO: 15).

This molecule can be seen in FIG. 1D. CPPtat is represented by SEQ ID NO: 2. An example of a decoy molecule can comprise amino acids 26-41 of SEQ ID NO: 1, and is represented by SEQ ID NO: 3, which includes a palmitoylation site at position 34. The full sequence of the decoy, including the CPP and palmitoylation site at position 34, is represented by SEQ ID NO: 4. Another example of a decoy molecule is represented by SEQ ID NO: 5, which includes a palmitoylation site at position 5. The full sequence of the decoy, including the CPP and palmitoylation site at position 5, is represented by SEQ ID NO: 6. Yet another example of a decoy molecule is represented by SEQ ID NO: 7, which includes a palmitoylation site at position 17. The full sequence of the decoy, including the CPP and palmitoylation site at position 17, is represented by SEQ ID NO: 8. Another example of a decoy molecule is represented by SEQ ID NO: 9, which includes a palmitoylation site at position 85. The full sequence of the decoy, including the CPP and palmitoylation site at position 85, is represented by SEQ ID NO: 10.

Also disclosed are proteins which are similar, but not identical, to SEQ ID NOS: 2-10. For example, disclosed herein is a molecule with 80, 81, 82, 83, 84, 85, 86, 87, 88, 89, 90, 91, 92, 93, 94, 95, 96, 97, 98, or 99% identity to any of SEQ ID NOs: 2-10. Put another way, disclosed herein is a molecule which is identical to any of SEQ ID NOS: 2-10, but has 1, 2, 3, 4, 5, or more residues which have been mutated but which allow the molecule to still function as a decoy.

Also disclosed herein are other methods of reversing, removing, or rendered disabled palmitoylation after it has been added to flotillin-1. This can be done enzymatically, or by slow hydrolysis of a thioester of palmitate. In one example, palmitoylation can be reversed, removed, or rendered disabled by removal of palmitate, such as by soluble acyl thioesterase. This can also be done by inhibiting enzymes which catalyze palmitoylation. For example, the enzyme can be a palmitoyltransferase, such as zDHHC-palmitoyl acyltransferase.

Other examples of tools that can be used to manipulate palmitoylation can be found in Main et al. (Protein S-Palmitoylation: advances and challenges in studying a therapeutically important lipid modification. FEBS J. 2022 February; 289(4):861-882. doi: 10.1111/febs.15781. Epub 2021 Mar. 18. PMID: 33624421), hereby incorporated by reference in its entirety. Examples include, but are not limited to, the general zDHHC-PAT inhibitor 2-bromopalmitate (2-BP). Often referred to as a ‘suicide inhibitor’, 2-BP, can irreversibly alter the zDHHC active site cysteine through nucleophilic displacement and alkylation. Additionally, lipid-based alternatives such as antibiotics tunicamycin, which has been shown to inhibit the palmitoylation of calcium channels and presynaptic plasticity protein GAP-43, and cerulenin, which has been reported to inhibit palmitoylation of fatty acid uptake channel CD36, can be used. Also disclosed is targeting an enzyme which is specific for palmitoylation of flotillin-1.

Inhibition of flotillin-1 can occur via gene knockout, or by small molecule, or by siRNA. For example the present invention provides an interfering RNA that silences (e.g., partially or completely inhibits) expression of a gene of interest (i.e., a palmitoyl acyl transferase gene). An interfering RNA can be provided in several forms. For example, an interfering RNA can be provided as one or more isolated small-interfering RNA (siRNA) duplexes, longer double-stranded RNA (dsRNA), or as siRNA or dsRNA transcribed from a transcriptional cassette in a DNA plasmid. The interfering RNA may also be chemically synthesized. The interfering RNA can be administered alone or co-administered (i.e., concurrently or consecutively) with conventional agents used to treat a bladder infection.

In one aspect, the interfering RNA is an siRNA molecule that is capable of silencing expression of a target sequence such as a palmitoyl acyl transferases sequence. In some embodiments, the siRNA molecules are about 15 to 60 nucleotides in length. The synthesized or transcribed siRNA can have 3′ overhangs of about 1-4 nucleotides, preferably of about 2-3 nucleotides, and 5′ phosphate termini. In some embodiments, the siRNA lacks terminal phosphates.

Suitable siRNA sequences can be identified using any means known in the art. Typically, the methods described in Elbashir et al, Nature, 411:494-498 (2001) and Elbashir et al, EMBO J, 20:6877-6888 (2001) are combined with rational design rules set forth in Reynolds et al, Nature Biotech., 22(3):326-330 (2004), for example.

Also disclosed herein are methods of identifying inhibitors of palmitoylation of flotillin-1, the method comprising the steps of providing flotillin-1; providing one or more enzymes which catalyze palmitoylation of flotillin-1; providing one or more potential inhibitors of flotillin-1 palmitoylation; and detecting inhibition of palmitoylation of flotillin-1 by a potential inhibitor, thereby identifying an inhibitor of palmitoylation of flotillin-1. This assay can be done in a high-throughput manner. technique for high throughput screening of compounds is described in PCT Application WO 84/03564. Large numbers of small peptide test compounds can be synthesized on a solid substrate, such as plastic pins or some other surface. Bound polypeptide is detected by various methods.

Lastly, it should be understood that while the present disclosure has been provided in detail with respect to certain illustrative and specific aspects thereof, it should not be considered limited to such, as numerous modifications are possible without departing from the broad spirit and scope of the present disclosure as defined in the appended claims.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

### EXAMPLES

### Example 1: Flotillin-1 Palmitoylation is Essential for its Stability and Subsequent Tumor Promoting Capabilities

### Background

Triple Negative Breast cancer (TNBC) is an aggressive subtype of breast cancer characterized by its high propensity to metastasize (Desantis 2015). Though there has been success in targeted therapies for other subtypes of breast cancer, TNBC therapies are mainly limited to highly cytotoxic chemotherapeutic approaches (Desantis 2015). Thus, there is a great need to uncover novel therapeutic targets in TNBC as well as mechanisms of metastasis, of which results in over 90% of TNBC associated mortality (Desantis 2015).

Flotillins are a class of proteins enriched in lipid rafts within plasma membranes (Amaddii 2012; Koh 205; Morrow 2002). Flotilln-1 and 2 often hetero-oligomerize with one another and offer stable signaling platforms for receptors in addition to promoting their intracellular trafficking, stability, and signaling (Morrow 2002). Recently, it has been found that flotillins contribute to a protein trafficking process termed upregulated flotillin-induced trafficking (UFIT), which involves aberrant plasma membrane invaginations that contribute to exacerbated receptor signaling from non-degradative late endosomal compartments (Genest 2020; Planchon 2018).

Flotillin expression is often lower in normal cells, however it is upregulated in many solid tumors (Ou 2017). Moreover, its expression is associated with invasion and metastasis (Ou 2017). Various findings have implicated flotillin-1 as a biomarker for lymph node metastasis including one study investigating flotillin-1 expression in lung adenocarcinoma through the utilization of unbiased membrane proteomics (Li 2016; Zhang 2012). Flotillin upregulation in numerous cancers has been associated with transforming growth factor beta (TGF-β) (Cao 2016), Axl receptor tyrosine kinase (Genest 2020), and Insulin-like growth factor-1 receptor (IGF-1R) signaling (Jang 2015; Dam 2020), all of which contribute to metastasis.

Flotillin-1 is modified by SUMOylation, phosphorylation, and s-palmitoylation (Jang 2015; Jang 2019: Riento 2009). S-palmitoylation is a reversible protein modification in which palmitoyl-CoA is covalently attached to cysteine residues through the formation of a thioester bond (Wang 2020). This process is catalyzed by a class of zDHHC palmitoyl acyl transferase enzymes (Wang 2020). The transfer of palmitoyl-CoA onto proteins through zDHHC enzymes alters their stability, trafficking, and processing (Coleman 2016; Ernst 2018). Given the strong evidence that flotillin-1 expression contributes to cancer metastasis, it was determined whether palmitoylation could be a contributing factor in its metastasis-inducing capabilities.

By constructing MDA-MB-231 TNBC cell lines to stably express a palmitoylation-defective form of flotillin-1, it was demonstrated that fotillin-1 palmitoylation is essential for its stability as well as its capacity to promote tumor growth and lung metastasis in vivo. Further investigation led to the discovery of zDHHC5 as the main palmitoyl acyl transferase responsible for palmitoylating endogenous flotillin-1, which contributes to an increase in its stability. For potential clinical relevance, a competitive palmtioylation inhibiting peptide was designed, and proof of concept was provided that indicates flotilln-1 palmitoylation as an actionable therapeutic target in TNBC.

### Materials and Methods

### Cell Culture

MDA-MB-231, SUM-159, and HEK 293T were purchased from ATCC and cultured in RPMI+10% FBS+1% penicillin/streptomycin. All cells were tested for mycoplasma by qPCR test. Cells were grown in an incubator at 37° C.+5% CO₂ with maintained humidity.

### Animal Experiments

All experiments were conducted within AAALAC guidelines and were approved by the University of Texas Institutional Animal Care and Use Committee (Protocol No. AUP-2022-00082). Six- to eight-week-old NOD/SCID mice (Charles River laboratories) were injected bilaterally with 1×10⁵ WT or C34A flotillin-1 expressing MDA-MB-231 cells also labelled with GFP and dTomato reporters, respectively. The cells were suspended in a 1:1 ratio of PBS to Matrigel and injected orthotopically into the 4^{th} mammary fat pad. Tumor diameter and mouse weight was monitored bi-weekly to ensure there were no significant changes in weight or that the tumor diameter did not exceed 1.5 cm. After 8-weeks of growth, animals were euthanized, and tissues were processed for imaging as described in the immunofluorescence section. For experimental lung metastasis experiments, 0.5×10⁶ MDA-MB-231 cells expressing flotillin-1 C34A or WT constructs along with a luciferase reporter lentiviral vector were injected into the lateral tail vein of six- to eight-week-old NOD/SCID mice (Charles River laboratories). Mice were injected with D-luciferin and anesthetized with isoflurane prior to BLI images using the IVIS. Images were taken at day 0, 7, 14, and 21 in mice. Lungs were then removed and placed in PBS containing D-luciferin for Ex Vivo lung imaging on day 28.

### Fluorescence Activated Cell Sorting

Primary tumors were minced with a scalpel and placed in DMEM containing 2 mg/mL of collagenase A +100 units of DNase I. Tumors were digested at 37° C. for three hours and then passed through a 70 μM nylon cell strainer to create a single cell suspension. The cell suspension was then spun down and re-suspended in PBS+2% FBS and 0.1% sodium azide. Gating strategies are reported in the supplemental information section.

### Plasmid Constructs, Cloning, Mutagenesis

To generate pFlotillin-1-GFP and pFlotillin-1-Cysteine-34-Alanine-GFP constructs, flotillin-1 full-length mRNA (NM_005803.4) was reverse transcribed using the Ultra script 2.0 cDNA synthesis kit (PCR Biosystems, #PB30.32-02). The full-length cDNA was then amplified with Phusion Plus Polymerase master mix (Thermo Scientific, #F631S) and primers according to manufactures instructions. Fragment 1 was amplified for the wild type flotillin-1 and two fragments for the C34A mutant both from full-length cDNA. For flotillin-1 C34A, the first fragment contained the mutation at cysteine 34 and the second fragment contained the remaining flotillin-1 CDS. Both wild type and C34A fragments were confirmed by gel electrophoresis and purified along with the HIND III and PstI digested pEGFP-SF2 vector. PCR fragments along with the digested vectors were subjected to the HIFI DNA assembly reaction (New England Biolabs, #E2621S) according to manufactures instructions. 2 ul from each reaction was transformed into DH5a competent cells (New England Biolabs, #C2987I). For generation of lentiviral constructs, the pFlotillin-1-GFP or pFlotillin-1-C34A-GFP constructs were used as templates for PCR using specific primers. Flotillin-1-GFP, flotillin-1-C34A, or dTomato constructs were amplified by PCR and cloned into a xbaI-SalI digested pCMV-GFP-hygro vector (Addgene #17446) by HIFI DNA assembly to create flotillin-1-GFP and flotillin-1-C34A-dTomato lentiviral constructs. For GFP-F1 peptide constructs, gBlocks from Integrated DNA technologies were synthesized to represent the CDS DNA sequence encoding amino acids 23-41 of the flotillin-1. This synthetic DNA also harbored 40 bp homology ends to XbaI-BamHI digested pCMV-hygro vector (Addgene, #17446). For all assembly reactions, either synthetic DNA gBlocks or PCR amplified fragments were combined with gel purified digested vectors, incubated at 50° C. with the HIFI DNA assembly reaction mix for 1 hour and transformed into NEB stable (New England Biolabs, #C3040I) competent cells. All plasmid sequences were confirmed by sanger sequencing and colony PCR.

### Transfections

Plasmids were transiently transfected using lipofectamine 3000 according to manufactures instructions. For 6 well dishes, 2 ug of plasmid was used and 10 ug for 100 mm plates. Media was replaced after 6 hours of transfection and protein expression was analyzed 48 hours post-transfection.

### Lentiviral Generation and Stable Cell Line Generation

To generate lentiviral particles Sug of transfer lentiviral vector was combined with 2^{nd} generation packaging plasmids, 3.3 ug pPAX.2 and 1.7 ug pMD2.g in sterile, nuclease free water up to 500 ul. 62 ul of 2M CaCl₂) was added to the plasmid mix. 500 ul of 2×Hepes buffer saline (HBS) was then added in a dropwise fashion and the tube was mixed vigorously. 100 mm dishes of HEK 293T cells were incubated for one hour with 25 uM chloroquine in DMEM before being transduced with the above mixture. Cells were transfected with the mixture for 7-12 hours after which the medium was replaced. 24 hours after transduction, fresh medium was applied, and viral supernatant collected after an additional 24 hours. and filtered through 0.45 μM filter. Filtered supernatant was incubated with lenti-pac lentivirus concentrator solution (genecopoeia, #LT007) at a 4:1 ratio and kept on ice for 3 hours. The viral pellet was then suspended in 100 ul of PBS and aliquoted for storage at −80 until further use. Cells were then seeded into 6-well plates and medium was replaced with DMEM containing 8 ug/mL of polybrene. To generate flotillin-1 WT and C34A stable cell lines, cells were first transduced with flotillin-1 3′ UTR shRNA particles and selected with puromycin (2 ug/ml). Once the cell lines were stable, flotillin-1 deficient cells were transduced with either flotillin-1-GFP or flotillin-1-C34A-dTomato lentiviral particles and selected with hygromycin (400 μg/ml) for at least two weeks. These cells were transduced to express additional flotillin-1 independent GFP or dTomato proteins from empty lentiviral vectors for cell tracking in vivo. Blasticidin (5 mg/ml) was used to select for stable integration. For tail vein metastasis, flotillin-1-GFP or flotillln-1 C34A cells were transduced with luciferase lentiviral particles and selected with blasticidin prior to tail vein injection.

### Immunofluorescence

For in vitro Immunofluorescence, cells were seeded on 8-well chamber slides and the following day fixed with 3.7% paraformaldehyde at room temperature for 15 minutes. Following fixation cells were permeabilized with 0.5% triton-x-100 for 20 minutes and blocked in blocking buffer (10% FBS, 2% BSA, 0.2% triton-x-100) for one hour at room temperature. Primary antibodies were diluted in dilution buffer (2% FBS, 2% BSA, 0.2% triton-x-100) and incubated overnight at 4° C. Slides were then washed three times in PBS for 5 minutes each and incubated with secondary antibodies in dilution buffer for 2 hours at room temperature. Slides were washed three times in PBS and mounted in prolong gold antifade medium containing DAPI. For in vivo immunofluorescence, primary tumors were incubated in 4% paraformaldehyde, while lungs 1% paraformaldehyde for 6 and 4 hours at 4° C., respectively. Fixed tissues were then placed in 25% sucrose overnight at 4° C. Tissues were embedded in O.C.T and frozen at −80° C. Frozen tissues were cut into 20 μM section, placed on super frost gold slides, and allowed to dry for 4 hours to overnight. O.C.T was removed by washing three times in PBS and permeabilized for 1 hour in 0.5% triton-X-100. Permeabilized slides were blocked at room temperature for two hours before being incubated with primary antibodies overnight at 4° C. Slides were washed three times for 5 minutes each in PBS before being incubated with secondary antibodies in dilution buffer for 2 hours at room temperature. Slides were then washed three times for 5 minutes each in PBS. Slides were mounted in mounting medium containing DAPI.

### Immunohistochemistry

To detect Ki67 and cleaved-caspase 3 in fixed-frozen tissue sections, 5 μM frozen tissue sections on super frost gold slides were allowed to thaw at room temperature for 30 minutes to an hour prior to being washed in PBS three times for 5 minutes each. Washed slides were then blocked in 0.3% H202 solution in PBS for ten minutes to block endogenous peroxidases. Following two PBS washes, the slides were then blocked for 2 hours at room temperature in the same antibody blocking buffer as the previous immunofluorescence section. Following the blocking step, slides were placed in a modified humidity chamber and incubated overnight at 4° C. with Ki67 (1:100) or Cleaved-Caspase 3 (1:100) antibodies diluted in the same antibody dilution buffer as the previous Immunofluorescence section. Following primary antibody incubation, slides were washed three times in PBS for 5 minutes each and incubated with biotinylated rabbit IgG (1:100) for 30 minutes at room temperature. The slides were again washed as described previously and incubated with streptavidin-HRP for an additional 30 minutes. After two additional PBS washes for 5 minutes each, the slides were incubate with diluted DAB substrate according to manufacturer's instructions until signal development. The slides were then washed with PBS and subjected to hematoxylin staining for 2 minutes followed by washing with tap water for 15 minutes. Washed slides were then dehydrated in a gradient ethanol bath (95%, 95%, 100%, 100%) for 5 minutes each. Slides were then cleared in xylene and mounted with aqueous mounting medium.

### Ubiquitylation and Protein Stability Detection

To detect for poly-ubiquitylation, cells were lysed in 1% SDS lysis buffer supplemented with or without tandem ubiquitin binding entities (TUBE) conjugated to biotin (Life Sensors, #UM-0301-0200) at a final concentration of 100 μg/ml. Lysates were incubated for 15 minutes at 4° C. and centrifuged for 10 minutes at 10,000 rpm. Supernatants were normalized to 100 μg of protein, diluted 10-fold in PBS and enriched overnight with strep-avidin magnetic beads. Beads were then washed three times with 1% SDS in PBS, suspended in 4× sample loading buffer, and heated at 95° C. for ten minutes before being subjected to SDS-PAGE. For protein stability, cells were seeded into 6-well plates and incubated with 100 μg/ml of cycloheximide for the desired time points before being lysed in 1% SDS lysis buffer. Additional rescue experiments included a cycloheximide treatment 100 μg/ml with MG-132 (20 μM) or chloroquine (25 μM) for 6 hours prior to being harvested with 1% SDS lysis buffer.

### 3D Culture and Invasion

To generate 3D spheroids, MDA-MB-231 and SUM 159 stable and parental cells were diluted to 1×10⁶/ml and pipetted onto the top of a 100 mm dish at 30 ul per drop. The top was then placed onto the bottom of the dish containing 5 ml of phosphate buffered saline (PBS). Spheroids were grown as hanging drop cultures for 48 hours prior to embedding into Cultrex Rat Collagen I (R&D Systems, #3440-100-01). To collect the spheroids, media was gently washed on the top of the dishes and spheroids were spun down in BSA coated tubes for 5 minutes at 400×g. 8-well chamber glass slides were coated by adding 75 ul of neutralized Collagen I to each well and left in the incubator overnight at 37° C. Collagen I was diluted to 2 mg/mL in serum free medium, neutralized with 1N NaOH, and allowed to polymerize on ice for 30 minutes. After polymerized, spheroids were mixed with the collagen I solution and added to each well at 250 ul per well. Embedded spheroids were allowed to solidify for 1 hour prior to the addition of full growth medium. Spheroids were allowed to grow for 5 days with medium being replaced every 48 hours. Spheroids were then fixed in 3.7% Paraformaldehyde for 15 minutes and washed three time in PBS for ten minutes each. Spheroids were imaged using an inverted phase contrast microscope. Invasive spheroids were classified as having two or more protrusions and non-invasive as having no protrusions.

### Invasion Chamber

Due to difficulty of transient transfection, only stable SUM 159 cells were used for invasion chamber analysis. shFlotillin-1 or shControl MDA-MB-231 cells were transiently transfected with lipofectamine 3000 as previously described with pCMV-GFP (empty vector), pFlotillin-1-GFP, or pFlotillin-1-C34A-GFP constructs for 48 hours prior to being trypsinized and counted. Cells were then diluted to 1×10⁶ cells/ml in serum-free medium. 24-well invasion chamber well inserts were pre-coated with Collagen I (2 mg/mL) for 24 hours at 37° C. for polymerization. 500 μL of full-growth medium was then added to the bottom of each well as a chemoattractant and 300 ul of each cell suspension in serum-free medium added to the collagen I coated insert. The remaining cells for each condition were then spun down at 10,000 rpm for 5 minutes, washed with PBS, and lysed for western blot analysis to confirm protein expression from respected constructs.

Diluted cells were then added to the inserts (300 ul per well) and incubated for 24 hours. The following day, medium was aspirated from the inserts and fixed with 3.7% paraformaldehyde for two minutes. The inserts were then washed with PBS once and permeabilized with 100% methanol for 20 minutes. After permeabilization, the inserts were washed in PBS and stained with Crystal Violet for an additional 20 minutes. Inserts were washed an additional two times in PBS. After washing, the top of the inserts (non-invaded cells) were removed with an cotton swab. Invaded cells were then imaged using an inverted microscope at random regions of interest. To ensure that regions of interest were not biased, invasion was quantified by de-staining the entire bottom insert with 10% acetic acid for 20 minutes at room temperature. The distaining solution was then transferred to a 96 well plate and read at 570 OD for an unbiased quantification of invasion.

### Western Blotting

Protein lysates were lysed in a 1% SDS lysis buffer containing 2 mM EDTA and 20 mM Tris-HCl (PH 6.8) for 5 minutes and scraped into 1.5 mL collection tubes. Lysates were sonicated using horn sonification for 3×20 seconds each and allowed to recover on ice in between each sonification. Lysates were centrifuged for 5 minutes at 10,000 rpm at 4° C. and supernatants subjected to protein quantification by BCA assay. Lysates were then diluted to 50 ug, resolubilized in 4×SDS loading buffer (200 mM Tris-HCL, 6.8 PH, 8% SDS, 40% glycerol, 0.4% bromophenol blue) and boiled at 95° C. for 7 minutes. Boiled samples were resolved on a 10% SDS-PAGE gel and transferred to a nitrocellulose membrane. Transferred membranes were blocked for 1 hour in 5% non-fat dairy milk (NFDM). Blocked membranes were washed with 1× tris buffer saline+0.1% tween-20 (TBST) prior to the addition of the diluted primary antibody overnight at 4° C. The following day, membranes were washed thrice with TBST for 5 minutes each prior to the addition of rabbit (1:3000) (Cell Signaling Technologies, #7074) or mouse IgG (1:5000) secondary horse radish peroxidase (HRP) conjugated antibody diluted in 5% NFDM for 1 hour. Membranes were washed thrice in TBST and developed by the addition of West Super Atto substrate. Images were obtained using an Sygene Imaging system.

### Click Chemistry

For click chemistry analysis, cells were grown to 80% confluence in 100 mm dishes and replaced with serum-free medium containing 25 μM of 17-Octodecanoyic Acid (17-ODYA) (Cayman Chemicals, #90270) or DMSO for 8 hours. Cells were lysed in 1% SDS lysis buffer+50 mM Tris-HCL (1×SDS buffer). Cells were incubated in 500 ul of buffer on ice for 30 minutes and transferred to 1.5 mL tubes. Lysates were sonicated with a probe sonicator for 3×20 seconds on ice. Lysates were centrifuged 10,000 rpm for 5 minutes and supernatants subjected to BCA assay (Thermo Scientific, #23227) for protein concentration. Lysates were diluted to 1 mg/ml and chloroform/methanol precipitated. Cell pellets were resolubilized in 50 ul of 1×SDS buffer and subjected to click chemistry reaction with click chemistry protein reaction kit (Click Chemistry Tools, #1262). Briefly, 50 ul of lysate was added to reaction buffer containing 40 μM of Azide Biotin (Click Chemistry Tools, #1265-5). The reaction was incubated for 30 minutes with rotation at room temperature protected from light. Excess Biotin was removed by an additional chloroform/methanol precipitation. Protein pellets were again resolubilized with 100 ul of 1×SDS buffer. 10 ul were kept as a 10% input and the remaining 90 ul were diluted 10× in PBS prior to being incubated with pre-washed Pierce streptavidin agarose (Thermo Scientific, #20349) (20 ul per sample) overnight (8-12 hours) with rotation at 4° C. The following day lysates were washed three times with 1×SDS buffer. Resins were then resolubilized in 15 ul of 1×SDS lysis buffer and heated to 95° C. for 10 minutes to elute proteins from resin. Supernatants were resolved on a 10% SDS-PAGE and subjected to western blot analysis.

### Acyl-Biotin Exchange

For acyl-biotin exchange, cells were treated with DMSO, TVB-3664 (100 nM)+BSA, or TVB-3664 (100 nM)+100 μM BSA-Palmitate conjugate (Please see ( ) for detailed palmitate conjugation protocol) in serum free medium for 48 hours and lysed with 1 mL of lysis buffer containing (100 mM Tris-HCL, PH 7.2, 5 mM EDTA, 150 mM NaCl, 2.5% SDS, and 50 mM of N-ethylmaleimide (NEM) to block free-cysteines. Samples were sonicated 3×20 seconds using a probe sonicator and incubated for 2 hours at 4° C. with rotation. After incubation, lysates were centrifuged for 10 minutes at 16,000 g. Supernatants were subjected to BCA protein assay for quantification of protein concentration. Lysates were diluted to 1 mg/mL and precipitated by chloroform/methanol precipitation. Precipitated protein was resolubilized in 500 ul of lysis buffer containing 0.5 mM of HPDP-Biotin (Cayman Chemicals, #16459) and divided into two parts. One part was incubated with 500 ul of 1M NaCl (—NH₂OH) and the other part with 1M Hydroxylamine (+NH₂OH) (Millipore Sigma, #438227) for 3 hours at room temperature protected from light with rotation. Lysates precipitated by chloroform/methanol and resolubilized in 200 ul of re-solubilization buffer (100 mM Tris-HCL 7.2 PH, 2% SDS, 8M Urea, 5 mM EDTA). 20 ul from each 200 ul sample was used as an input with the remaining sample diluted 10× with PBS. 20 ul of pre-washed streptavidin agarose (Thermo Scientific, #20349) was added to each lysate and incubated overnight at 4° C. with rotation. Lysates were then washed three times with 1% SDS in PBS. Resins were then solubilized in 15 ul of 4×SDS loading buffer and eluted by boiling at 95° C. for 10 minutes. Supernatants were resolved on a 10% SDS-PAGE and subjected to western blot.

### Peptide Treatments

CPP and CPP-F1 peptides were synthesized by ABclonal and delivered as a lyophilized powder. The peptides were re-suspended in sterile nuclease-free water to make a concentrated stock of 1200 uM and 500 uM, respectively. The peptides were then aliquoted and stored at −80 protected from light until use. Peptides were added to culture medium at desired concentrations for 24 hours for in vitro treatments.

### REFERENCES

1. Desantis, C. E., et al., Breast cancer statistics, 2019. CA: A Cancer Journal for Clinicians, 2019. 69(6): p. 438-451.
2. Jang, D., et al., Essential role of flotillin-1 palmitoylation in the intracellular localization and signaling function of IGF-1 receptor. J Cell Sci, 2015. 128(11): p. 2179-2190.
3. Amaddii, M., et al., Flotillin-1/reggie-2 protein plays dual role in activation of receptor-tyrosine kinase/mitogen-activated protein kinase signaling. Journal of Biological Chemistry, 2012. 287(10): p. 7265-7278.
4. Koh, M., et al., A novel role for flotillin-1 in H-R as-regulated breast cancer aggressiveness. International journal of cancer, 2016. 138(5): p. 1232-1245.
5. Morrow, I. C., et al., Flotillin-1/Reggie-2 Traffics to Surface Raft Domains via a Novel Golgi-independent Pathway: IDENTIFICATION OF A NOVEL MEMBRANE TARGETING DOMAIN AND A ROLE FOR PALMITOYLATION. 2002. 277(50): p. 48834-48841.
6. Genest, M., et al., Flotillin-upregulation acts as an epithelial-mesenchymal transition driver by promoting sphingosine kinase 2-dependent AXL stabilization. bioRxiv, 2020: p. 2020.05. 12.090571.
7. Planchon, D., et al., MT1-MMP targeting to endolysosomes is mediated by upregulation of flotillins. Journal of cell science, 2018. 131(17).
8. Ou, Y., et al., Prognostic value of Flotillin-1 expression in patients with solid tumors. Oncotarget, 2017.
9. Li, Z., et al., Elevated expression of flotillin-1 is associated with lymph node metastasis and poor prognosis in early-stage cervical cancer. American journal of cancer research, 2016. 6(1): p. 38.
10. Zhang, P.-F., et al., Identification of flotillin-1 as a novel biomarker for lymph node metastasis and prognosis of lung adenocarcinoma by quantitative plasma membrane proteome analysis. Journal of proteomics, 2012. 77: p. 202-214.
11. Cao, S., et al., Upregulation of flotillin-1 promotes invasion and metastasis by activating TGF-β signaling in nasopharyngeal carcinoma. Oncotarget, 2016. 7(4): p. 4252.
12. Dam, D. H. M., et al., Flotillin and AP2A1/2 Promote IGF-1 Receptor Association with Clathrin and Internalization in Primary Human Keratinocytes. Journal of Investigative Dermatology, 2020.
13. Jang, D., et al., Sumoylation of Flotillin-1 promotes EMT in metastatic prostate cancer by suppressing Snail degradation. Oncogene, 2019.
14. Riento, K., et al., Endocytosis of flotillin-1 and flotillin-2 is regulated by Fyn kinase. Journal of cell science, 2009. 122(7): p. 912-918.
15. Wang, Y., et al., Palmitoylation as a Signal for Delivery. 2020, Springer Singapore. p. 399-424.
16. Coleman, D. T., et al., Palmitoylation regulates the intracellular trafficking and stability of c-Met. Oncotarget, 2016. 7(22): p. 32664.
17. Ernst, A. M., et al., S-Palmitoylation Sorts Membrane Cargo for Anterograde Transport in the Golgi. Developmental Cell, 2018. 47(4): p. 479-493.e7.
18. Weigelt, B., et al. (2005). “Breast cancer metastasis: markers and models.” Nature Reviews Cancer 5(8): 591-602.
19. Yao, H., et al. (2019). “Inhibiting PD-L1 palmitoylation enhances T-cell immune responses against tumours.” Nature biomedical engineering 3(4): 306-317.

### TABLES

TABLE 1
Various Cysteine Residues in the Flotillin-1 Gene for use as
Decoy Palmitoylation Sites

| | | | Code for Sequence |
|---|---|---|---|
| | | | in Flotillin-1 |
| | | Peptide Sequence | FASTA Sequence |
| Cysteine | Protein | (Palmitoylation | (SEQ ID NO: 1, |
| Location | Domain | Site Underlined) | below) |
| C5 | SPFH/PHB | MFFTCGPNEAMV | Underlined (AMV |
| | | (SEQ ID NO: 5) | overlap) |
| C17 | SPFH/PHB | AMVVSGFCRSPPVMV | Bolded (AMV |
| | | (SEQ ID NO: 7) | Overlap) |
| C34 | SPFH/PHB | GGR VFVLPCIQQIQRI | Underlined |
| | | (SEQ ID NO: 3) | |
| C85 | SPFH/PHB | KEMLAAACQMFLGKT | Bolded |
| | | (SEQ ID NO: 9) | |

SEQUENCES

SEQ ID NO: 1: Full length flotillin-1
MFFTCGPNEAMVVSGFCRSPPVMVAGGRVFVLPCIQQIQRISLNTLTLNVKSEK
VYTRHGVPISVTGIAQVKIQGQNKEMLAAACQMFLGKTEAEIAHIALETLEGH
QRAIMAHMTVEEIYKDRQKFSEQVFKVASSDLVNMGISVVSYTLKDIHDDQDYL
HSLGKARTAQVQKDARIGEAEAKRDAGIREAKAKQEKVSAQYLSEIEMAKAQR
DYELKKAAYDIEVNTRRAQADLAYQLQVAKTKQQIEEQRVQVQVVERAQQVA
VQEQEIARREKELEARVRKPAEAERYKLERLAEAEKSQLIMQAEAEAASVRMRG
EAEAFAIGARARAEAEQMAKKAEAFQLYQEAAQLDMLLEKLPQVAEEISGPLTS
ANKITLVSSGSGTMGAAKVTGEVLDILTRLPESVERLTGVSISQVNHKPLRTA
SEQ ID NO: 2 example CPPtat sequence
YGRKKRRQRRR
SEQ ID NO: 3: Decoy sequence with palmitoylation site 34
GGRVFVLPCIQQIQRI
SEQ ID NO: 4 Decoy sequence with CPP and palmitoylation site 34
YGRKKRRQRRR GGRVFVLPCIQQIQRI
SEQ ID NO: 5 Decoy sequence using palmitoylation site 5
MFFTCGPNEAMV
SEQ ID NO: 6: Decoy sequence with CPP and palmitoylation site 5
YGRKKRRQRRR MFFTCGPNEAMV
SEQ ID NO: 7: Decoy sequence using palmitoylation site 17
AMVVSGFCRSPPVMV
SEQ ID NO: 8: Decoy sequence with CPP and palmitoylation site 17
YGRKKRRQRRR AMVVSGFCRSPPVMV
SEQ ID NO: 9: Decoy sequence using palmitoylation site 85
KEMLAAACQMFLGKT
SEQ ID NO: 10: Decoy sequence with CPP and palmitoylation site 85
YGRKKRRQRRR KEMLAAACQMFLGKT
SEQ ID NO: 11: TAT
GRKKRRQRRRPQ
SEQ ID NO: 12: Penetratin
RQIKIWFQNRRMKWKK
SEQ ID NO: 13: Pep-1
KETWWETWWTEWSQP-KKKRKV
SEQ ID NO: 14: MPG
GALFLGFLGAAGSTMGAWSQP-KKKRKV
SEQ ID NO: 15 Polyarginine (R9, R8)
RRRRRRRRR

1. A method of inhibiting flotillin-1 activity, the method comprising decreasing palmitoylation of flotillin-1 in a cell.

2. (canceled)

3. (canceled)

4. The method of claim 1, wherein decreasing palmitoylation decreases post-translational modifications of other proteins by flotillin-1.

5. The method of claim 1, wherein activity of flotillin-1 is decreased by at least 10% in the cell.

6. The method of claim 1, wherein decreasing palmitoylation of flotillin-1 decreases cancer metastasis or tumor growth in a subject.

7. (canceled)

8. The method of claim 1, wherein palmitoylation of flotillin-1 is decreased by providing a peptide inhibitor of flotillin-1.

9. The method of claim 8, wherein the competitive inhibitor comprises a cell penetrating peptide.

10. The method of claim 8, wherein the peptide inhibitor comprises a peptide which is a decoy for palmitoylation of position C5, C17, C34, or C85 of flotillin-1, wherein flotillin-1 is represented by SEQ ID NO: 1.

11. (canceled)

12. (canceled)

13. (canceled)

14. The method of claim 8, wherein the peptide inhibitor comprises a peptide which is a decoy for palmitoylation of at least two of these positions: C5, C17, C34, and C85 of flotillin-1, wherein flotillin-1 is represented by SEQ ID NO: 1.

15. The method of claim 8, wherein the peptide inhibitor comprises a peptide which is a decoy for palmitoylation of at least three of these positions: C5, C17, C34, and C85 of flotillin-1, wherein flotillin-1 is represented by SEQ ID NO: 1.

16. The method of claim 8, wherein the peptide inhibitor comprises a peptide which is a decoy for palmitoylation of all four of these positions: C5, C17, C34, and C85 of flotillin-1, wherein flotillin-1 is represented by SEQ ID NO: 1.

17. The method of claim 1, wherein palmitoylation is reversed, removed, or rendered disabled after its addition to flotillin-1.

18. The method of claim 17, wherein palmitoylation is reversed, removed, or rendered disabled enzymatically.

19. The method of claim 18, wherein palmitoylation is reversed, removed, or rendered disabled by slow hydrolysis of a thioester of palmitate or removal of palmitoyl.

20. (canceled)

21. The method of claim 19, wherein palmitoyl is removed by soluble acyl thioesterase.

22. The method of claim 1, wherein palmitoylation of flotillin-1 is decreased by inhibiting an enzyme or enzymes which catalyze palmitoylation.

23. The method of claim 22, wherein the enzyme is a palmitoyltransferase.

24. The method of claim 22, wherein the enzyme is a zDHHC-palmitoyl acyltransferase.

25. The method of claim 22, wherein the enzyme which specifically targets palmitoylation of flotillin-1 is inhibited.

26. The method of claim 25, wherein such inhibition occurs via gene knockout, small molecule, or siRNA.

27. (canceled)

28. (canceled)

29. A method of treating or preventing cancer, the method comprising decreasing palmitoylation of flotillin-1 in a subject with, or at risk of developing, cancer.

30-71. (canceled)
